(19) United States
(12) Patent Application Publication
(10) Pub. No.: US 2010/0210616 A1
(43) Pub. Date: Aug. 19, 2010

(54) METAL COMPLEXES INCORPORATED WITHIN BIODEGRADABLE NANOPARTICLES AND THEIR USE

(75) Inventors: Wiley J. Youngs, Akron, OH (US); Khadijah M. Hindi, Akron, OH (US); Douglas A. Medvetz, Elyria, OH (US); Matthew Panzner, Akron, OH (US); Claire Tessier, Akron, OH (US)

Correspondence Address: HAHN LOESER & PARKS, LLP, One GOJO Plaza, Suite 300, AKRON, OH, 44311-1076, US

(73) Assignee: THE UNIVERSITY OF AKRON, Akron, OH (US)

(21) Appl. No.: 12/670,141

(22) PCT Filed: Jul. 22, 2008
(86) PCT No.: PCT/US08/70697

§ 371 Date: Apr. 21, 2010

Related U.S. Application Data
(60) Provisional application No. 60/951,297, filed on Jul. 23, 2007.

(51) Int. Cl. A61K 31/555; C07F 1/10; A61P 31/10; A61P 31/04

(52) U.S. Cl. 514/186; 548/103; 544/225; 514/184

(57) ABSTRACT

Compounds for antimicrobial applications, and for treating bacterial and fungal infection are set forth. The compounds may include a metal complex incorporated into a biodegradable polymeric nanoparticle. Also, a method of treating bacterial and fungal infections in a mammal includes the steps of administering an effective amount of a silver(I) metal salt incorporated into a biodegradable polymeric nanoparticle.

### CROSS-REFERENCE TO RELATED APPLICATION

This is a national stage application of PCT/U.S.08/70697, filed Jul. 22, 2008, to which this application claims priority from and any other benefit of U.S. Provisional Application No. 60/951,297 filed on Jul. 23, 2007, the entire disclosure of which is hereby incorporated by reference.

### TECHNICAL FIELD

The invention relates to metal complexes which are used in treating bacterial and fungal infections. More particularly, the invention relates to metal complexes that are incorporated within biodegradable materials which are used in treating bacterial and fungal infections. Even more particularly, the invention relates to metal complexes that are incorporated within biodegradable nanoparticles which are used in treating bacterial and fungal infections.

### BACKGROUND OF THE INVENTION

Silver has long been used for its antimicrobial properties. This usage predates the scientific or medical understanding of its mechanism. For example, the ancient Greeks and Romans used silver coins to maintain the purity of water. Today silver is still used for this same purpose by NASA on its space shuttles. Treatment of a variety of medical conditions using silver nitrate was implemented before 1800. A 1% silver nitrate solution is still widely used today after delivery in infants to prevent gonorrheal ophthalmia. Since at least the later part of the nineteenth century, silver has been applied in a variety of different forms to treat and prevent numerous types of bacteria related afflictions.

Other treatments, such as the application of silver foil to post surgical wounds to prevent infection survived as a medical practice into the 1980's in Europe, and silver nitrate is still used as a topical antimicrobial agent. In the 1960's the very successful burn treatment silver complex, silver sulfadiazine, shown in formula 1 below, was developed. Commercially known as Silvadene® Cream 1%, this complex has remained one of the most effective treatments for preventing infection of second and third degree burns. Silver sulfadiazine has been shown to have good antimicrobial properties against a number of gram-positive and gram-negative bacteria. It is believed that the slow release of silver at the area of the superficial wound is responsible for the process of healing. Studies on surgically wounded rats have shown the effectiveness of both silver nitrate and silver sulfadiazine to aid in the healing process. By using these common silver antimicrobial agents, inflammation and granulation of wounds were reduced, although the complete mechanism for these phenomena is not understood.

One of the most common pulmonary ailments is pneumonia, which is the leading cause of death due to infectious causes in industrialized countries. Hospital-acquired pneumonia (HAP) and ventilator-associated pneumonia (VAP) are the leading causes of death from nosocomial infections affecting up to 25% of all intensive care unit patients with mortality rates as high as 70%. Typical therapy includes broad-spectrum intravenous antibiotics. However, organisms with multi-drug resistance cause a growing number of nosocomial infections leading to treatment failures. Since deposition in the lungs via the inhaled route can result in much higher drug concentrations than can be achieved through intravenous administration, delivery of existing antimicrobials via the inhaled route is appealing. The organisms responsible for HAP and VAP are varied, but include resistant strains of Pseudomonas and methicillin-resistant strains of Staphylococcus aureus (MRSA). These pathogens are also those seen in CF patients. Thus, novel antimicrobials active in the HAP and VAP patients are likely to have utility in treating CF patients, as well.

Respiratory infections, including pneumonia, are treated primarily with systemic antimicrobials, although recently, antimicrobials have been formulated for delivery directly to the lungs via nebulization. Although nebulized antimicrobials are accepted as a treatment modality for cystic fibrosis patients, they are not routinely used for treatment of acute pneumonia due to the availability of effective systemic therapy. However, with the advent of multi-drug resistant organisms, interest in nebulized antimicrobials as adjunct therapy has expanded. Pre-clinical animal studies have demonstrated increased efficacy of nebulized antimicrobials when impregnated into nanoparticles to achieve higher and more sustained concentrations.

Inhalation is a common technique of drug administration to patients with a variety of lung diseases. Besides anti-asthma drugs such as β2-agonist drugs, corticosteroids and anticholinergic drugs that are delivered via inhalation, antibiotics are the second most commonly delivered therapeutic agents delivered using this route, especially for the treatment of cystic fibrosis. The advantage of inhalation therapy for treatment of lung disease is that the drug is administered directly to the site of action and as a result, the lag time of the action onset of the drug is short, less therapeutic agent is needed and systemic side effects are reduced. Three different types of devices are commonly used for the administration of drugs to the respiratory tract: nebulizers, pressurized metered dose inhalers (pMDI) and dry powder inhalers (DPI).

Nebulizers are typically used to aerosolize drug solutions and sometimes drug suspensions for inhalation. This type of a system is predominantly used in situations where severe obstruction of the airways or insufficient coordination by the patient does not allow the use of other systems. One such category of patients is young children who cannot manage other devices. Furthermore, nebulizers are used for drugs such as antibiotics, antifungals, enzymes and mucolytic agents that cannot or have not been formulated with other device types such as pMDI and DPIs. Despite its advantages and widespread use in the delivery of 132-agonists and anticholinergic drugs, a drawback of inhalation therapy via nebulization is the low deposition efficiency of the drug in the target area. However, by devising an appropriate device and formulation and by developing a thorough understanding of the working principles of nebulization, drugs can be delivered over a wide range of doses in a very effective manner. In many instances with proper optimization of the inhalable composition, respirable fractions greater than about 50% can be realized. With this efficiency, aerosol delivery of drugs has become more attractive as first line therapy for common pulmonary ailments.

In recent years an increasing interest in the field of biodegradable polymers for their use as drug delivery systems has occurred. The majority of this research has included the biodegradable nanoparticles poly(glycolic acid) (PGA), poly(lactic acid) (PLA), and poly(lactic-co-glycolic acid) (PLGA) because they are approved by the FDA. PGA has been used in biodegradable suture materials since the 1970's.

Recent research has explored the loading of commercially available anticancer drugs, such as Paclitaxel (IUPAC name β-(benzoylamino)-α-hydroxy-,6,12b-bis(acetyloxy)-12-(benzoyloxy)-2a,3,4,4a,5,6,9,10,11,12,12a,12b-dodecahydro-4,11-dihydroxy-4a,8,13,13-tetramethyl-5-oxo-7,11-methano-1H-cyclodeca(3,4)benz(1,2-b)oxet-9-ylester,(2aR-(2a-α,4-β,4a-β,6-β,9-α(α-R*,β-S*),11-α,12-α,12a-α,2b-α))-benzenepropanoic acid), into PLGA nanoparticles for drug delivery. One of the drawbacks of this drug is its hydrophobicity which leads to a slow absorption of the drug into the body. However, the loading of Paclitaxel into PLGA has lead to increased efficacy. This is due mainly to the increase in hydrophilicity of the prepared nanoparticles.

Another existing drug delivery system used for biomedical applications is the polyaminophosphazenes with amino acid ester side chains. This class of compounds ultimately degrades into products that are bio-friendly, including phosphates and ammonia. The two main polyaminophosphazenes that have been used to date are poly(di(ethyl glycinato) phosphazene) (PEGP) and poly(di(ethyl alaninato) phosphazene) (PEAP).

Notwithstanding the state of the art as described herein, there is a need for further improvements in providing metal complexes that are incorporated within biodegradable nanoparticles which are used in treating bacterial and fungal infections.

### SUMMARY OF THE INVENTION

In general, one aspect of the invention is to provide a compound for treating bacterial and fungal infections, the compound comprising a metal complex incorporated into a polymeric or other nanoparticle.

Another aspect of the invention is to provide a metal complex for treating bacterial and fungal infections and for other antimicrobial applications wherein the metal complex is a silver(I) salt, a silver(I) macrocyclic metal complex, a silver(I) N-heterocyclic carbene or mixtures thereof.

In yet another aspect of the invention, the silver(I) macrocyclic metal complex is:

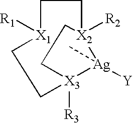

wherein each R is independently selected from the group consisting of a proton, an alkyl, an ether, an alcohol, a carboxylic acid, an aryl, an amino acid, a peptide, or null, wherein X₁, X₂ and X₃ are independently either sulfur or nitrogen, and when X₁, X₂ or X₃ is sulfur then R is null, wherein the macrocyclic ligand comprised of carbon, R₁₋₃, and X₁₋₃, represents L, wherein Y is selected from the group consisting of NO₃, OAc, SCN, BF₄, OTf, SO₄, Cl, Br, and I, or may represent L, and wherein Y represents L, then the counter anion is selected from the group consisting of NO₃⁻, OAc⁻, SCN⁻, BF₄⁻, OTf⁻, SO₄⁻, Cl⁻, Br⁻, and I⁻.

A further aspect of the invention, the silver(I) N-heterocyclic carbene is:

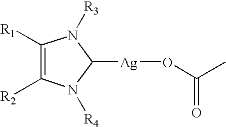

wherein R₁ and R₂ are selected from the group consisting of a halide, a proton, an alkyl, an ether, an alcohol, a nitro, a cyano, and a carboxylic acid, wherein R₃ and R₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

In yet a further aspect of the invention, the silver(I) N-heterocyclic carbene is:

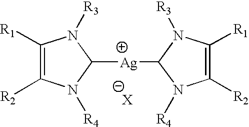

wherein R₁ and R₂ are selected from the group consisting of a halide, a proton, an alkyl, an ether, an alcohol, a nitro, a cyano, and a carboxylic acid, wherein R₃ and R₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

In another aspect of the invention, the silver(I) N-heterocyclic carbene is:

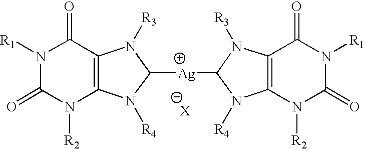

wherein R₁₋₄ can are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

In yet another aspect of the invention, a method of treating bacterial and fungal infections in a mammal includes the steps of:

administering an effective amount of a silver(I) metal salt incorporated into a biodegradable polymeric nanoparticle.

In another aspect of the invention, a method of treating bacterial and fungal infections in a mammal includes the steps of:

administering an effective amount of a macrocyclic silver(I) complex, the macrocyclic complex comprising:

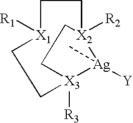

wherein each R is independently selected from the group consisting of a proton, an alkyl, an ether, an alcohol, a carboxylic acid, an aryl, an amino acid, a peptide, or null, wherein X₁, X₂ and X₃ are independently either sulfur or nitrogen, and when X₁, X₂ or X₃ is sulfur then R is null, wherein the macrocyclic ligand comprised of carbon, R₁₋₃, and X₁₋₃, represents L, wherein Y is selected from the group consisting of NO₃, OAc, SCN, BF₄, OTf, SO₄, Cl, Br, and I, or may represent L, and wherein Y represents L, then the counter anion is selected from the group consisting of NO₃⁻, OAc⁻, SCN⁻, BF₄⁻, OTf⁻, SO₄⁻, Cl⁻, Br⁻, and I⁻.

An aspect of the invention, a method of treating bacterial and fungal infections in a mammal includes the steps of:

administering an effective amount of a N-heterocyclic silver(I) complex, the N-heterocyclic complex comprising:

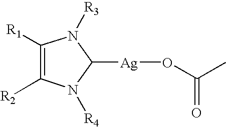

wherein R₁ and R₂ are selected from the group consisting of a halide, a proton, an alkyl, an ether, an alcohol, a nitro, a cyano, and a carboxylic acid, wherein R₃ and R₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

In another aspect of the invention, a method of treating bacterial and fungal infections in a mammal includes the steps of:

administering an effective amount of a N-heterocyclic silver(I) complex, the N-heterocyclic complex comprising:

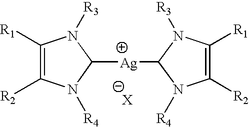

wherein R₁ and R₂ are selected from the group consisting of a halide, a proton, an alkyl, an ether, an alcohol, a nitro, a cyano, and a carboxylic acid, wherein R₃ and R₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

In yet another aspect of the invention, a method of treating bacterial and fungal infections in a mammal includes the steps of:

administering an effective amount of a N-heterocyclic silver(I) complex, the N-heterocyclic complex comprising:

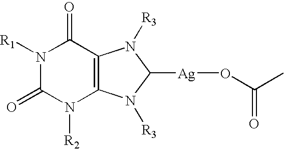

wherein R₁₋₄ can are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

In another aspect of the invention, a method of treating bacterial and fungal infections in a mammal includes the steps of:

administering an effective amount of a N-heterocyclic silver(I) complex, the N-heterocyclic complex comprising:

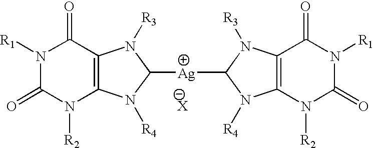

wherein R₁₋₄ can are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

### DETAILED DESCRIPTION OF THE DRAWINGS

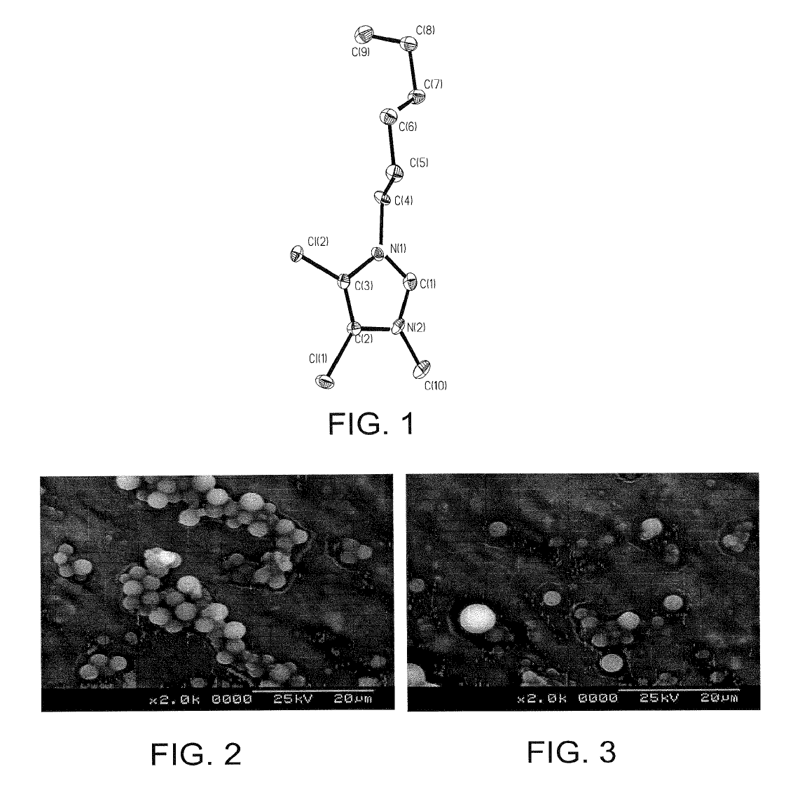

FIG. 1 is a representative crystal structure of the cationic portion of a 1-hexyl-3-methyl-4,5-dichloroimidazolium iodide salt precursor with thermal ellipsoids shown at 50% probability;

FIG. 2 is a scanning electron microscope image of silver(I) carbene complex of formula 17 loaded L-tyrosine polyphosphate nanospheres;

FIG. 3 is a scanning electron microscope image of blank L-tyrosine polyphosphate nanospheres;

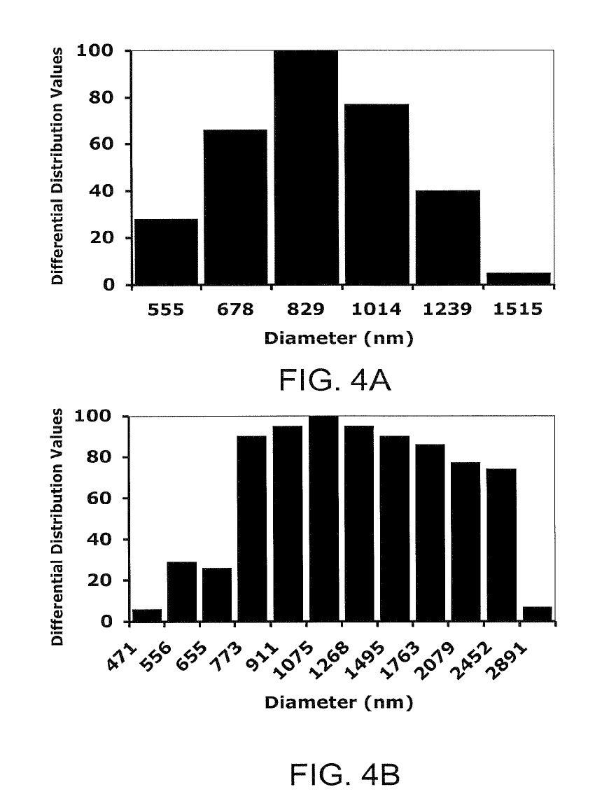

FIG. 4A is a graph representing the diameter distribution from dynamic laser light scattering of silver(I) carbene complex of formula 17-loaded L-tyrosine polyphosphate nanospheres;

FIG. 4B is a graph representing the diameter distribution from dynamic laser light scattering of blank L-tyrosine polyphosphate nanospheres;

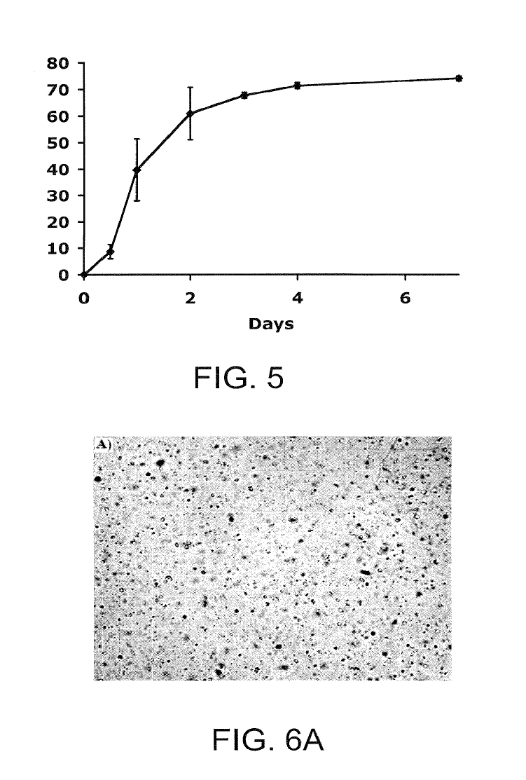

FIG. 5 is a graph representing the cumulative release of silver(I) carbene complex of formula 17 from about 1 mg of L-tyrosine polyphosphate nanospheres after incubation in PBS at 37° C. for 7 days;

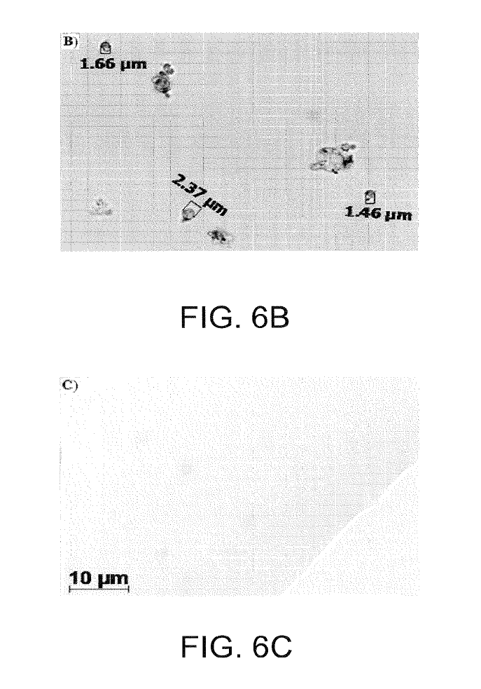

FIG. 6A is a light microscope gross image of L-tyrosine polyphosphate nanospheres after nebulization using 10× objective;

FIG. 6B is a light microscope gross image of L-tyrosine polyphosphate nanospheres after nebulization using 63× objective;

FIG. 6C is a light microscope gross image of L-tyrosine polyphosphate nanospheres in phosphate buffer after nebulization using 63× objective (nanospheres were not observed in this solution) with the air/water interface observable in the lower right portion of the figure;

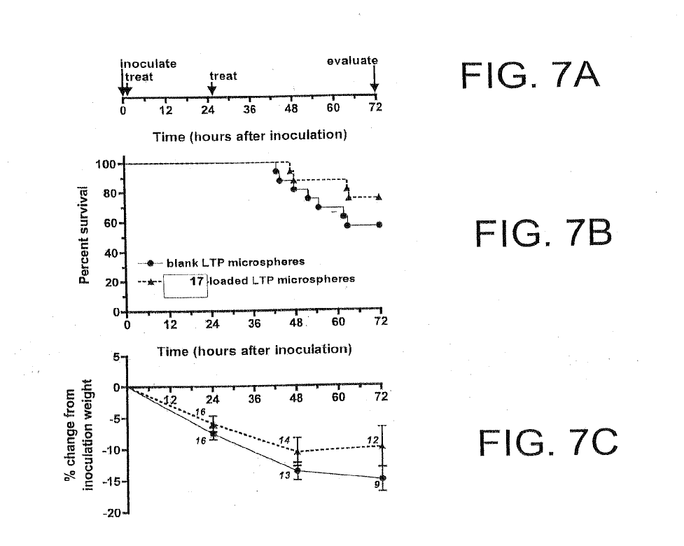

FIG. 7A represents the treatment protocol of mice receiving with nebulized blank or silver(I) carbene complex of formula 17-loaded L-tyrosine polyphosphate nanospheres one hour after infection with P. aeruginosa PA M57-15, and again 24 h after the first treatment, until mice were evaluated on day 3 (72 h);

FIG. 7B is a graph representing the Kaplan-Meier survival curves of mice treated with silver(I) carbene complex of formula 17-loaded L-tyrosine polyphosphate nanospheres compared with that of mice treated with blank nanospheres;

FIG. 7C is a graph representing the weight loss of mice treated with silver(I) carbene complex of formula 17-loaded L-tyrosine polyphosphate nanospheres compared with that of mice treated with blank nanospheres;

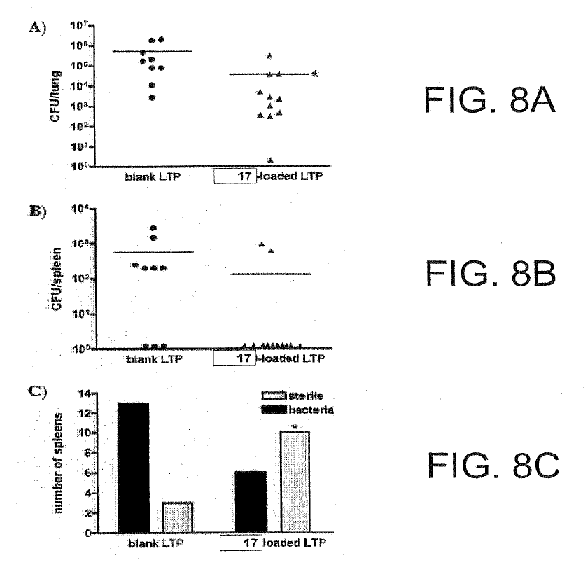

FIG. 8A is a graph representing the total number of bacteria recovered from the lungs of mice surviving at 72 h. after being nebulized with silver(I) carbene complex of formula 17-loaded L-tyrosine polyphosphate nanospheres and blank nanospheres;

FIG. 8B is a graph representing the total number of bacteremia as indicated by recovery of bacteria from the spleen after the indicated treatment in surviving mice analyzed at 72 h with the bar representing the mean bacterial count; and

FIG. 8C is a graph representing the total number of bacteremia in all of the mice plotted as number of spleens in each treatment group with (dark bars) or without (light bars) bacteria.

### DETAILED DESCRIPTION OF THE INVENTION

The use of metal compounds, including metal complexes, in conjunction with biodegradable nanoparticles for the antimicrobial applications, including treatment of bacterial and fungal infections, is disclosed. More specifically, the present invention includes, but is not limited to silver(I) metal complexes as simple salts, silver(I) macrocyclic metal complexes, and silver(I) N-heterocyclic carbenes (NHCs) incorporated within these biodegradable nanoparticles for antimicrobial applications, including for example, the treatment of bacterial and fungal infections.

Delivery of therapeutic agents such as proteins and DNA to the lungs via a pulmonary route shows great potential for the cure of several inherited and acquired diseases such as asthma, cystic fibrosis and lung cancer. Aerosol inhalation via a method like nebulization provides an easy and a non-invasive route to accomplish that objective. However, these agents are not amenable to conventional delivery formulations due to problems associated with loss of activity of the therapeutic agent during the nebulization process and by the action of enzymes and immune system components within the body. Under such circumstances, encapsulation of these agents in devices such as nanospheres, microspheres, liposomes, etc provides a means to protect the therapeutic agent during the delivery process and against the aggressive environmental factors, and may also enhance the agent's systemic bioavailability. The use of such a device can also help achieve a targeted and controlled delivery of the encapsulated pharmaceutical agent. Thus, the use of a controlled drug delivery device, such as polymeric nanoparticles in conjunction with a non-invasive method of delivery like nebulization has potential due to its ability to overcome current limitations associated with pulmonary delivery technology. The ability to achieve controlled release of a pharmacologically active agent to a specific site of action at the therapeutically optimal rate and dose regimen has been a major driving force in the development of such devices.

Nanoparticles generally vary in size from 10 nm to 1000 nm. These sub-micron sized particles possess certain distinct advantages over microparticles. Nanoparticles, including nanospheres, unlike microspheres, can be used to directly target the tissues via systemic circulation or across the mucosal membrane. This targeting is possible as a result of the capacity of these nanoparticles to be endocytosed by individual cells. It has also been observed that nanoparticles administered intravenously are taken up by cells of mononuclear phagocyte system, mainly in the Kuppfer cells. Such nanoparticles are rapidly cleared from the blood and are usually concentrated in the liver, spleen and blood marrow.

In the case of a nanoparticle type delivery system, the therapeutic agent is dissolved, encapsulated, entrapped or chemically conjugated to the nanoparticle matrix depending on the method of fabrication of the device. The drug may be physically and uniformly incorporated and dispersed within a nanosphere matrix. The drug formulated in such a polymeric device for example, is released by diffusion through the polymeric matrix, erosion of the polymeric matrix or by a combination of diffusion and polymer erosion mechanisms. In one embodiment of the invention, biodegradable, polymeric nanoparticles including poly(glycolic acid) (PGA), poly(lactic acid) (PLA), and poly(lactic-co-glycolic acid) (PLGA) are used.

Nanoparticles of this type provide controlled release of the antimicrobial materials in examples of the invention, and further have the ability to target particular organs and tissues and have ability to encapsulate and deliver material through a peroral route of administration.

Conventionally, the methods used to prepare nanoparticles can be broadly classified into two: (1) dispersion of the preformed polymers, and (2) polymerization of monomers, however; several different variations of each of the above methods may be used to optimize the product formulation. Variations of the first method that have been used to prepare nanoparticles for example include (a) solvent evaporation method, (b) spontaneous emulsification/solvent diffusion method and salting out/emulsification-diffusion method. In such approaches, the organic solvent is removed in a controlled manner thereby bringing about the precipitation of the polymeric particles. The encapsulation of the drug is carried out by dissolving the drug in the organic phase containing the polymer or an inner aqueous phase depending on the relative hydrophilicity and solubility of the material. In case of polymeric nanoparticles prepared by polymerization of monomers, the polymer usually has a lower solubility in the polymerization medium compared to the monomer. This results in the precipitation of the polymer with an increase in the molecular weight of the polymer. A control over the particle size is achieved by altering parameters such as rate of mechanical stirring, type and concentration of surfactant and/or stabilizer used, pH of the polymerization medium, etc. The material can be encapsulated within the nanoparticles either during the polymerization process or post-polymerization.

One example group of nanoparticles usable in the invention includes polyphosphazenes [PR₂N]ₙ. Polyphosphazenes are versatile polymers because they can be functionalized with a large variety of R groups by simply displacing the chlorides of the parent [PCl₂N]ₙ polymer. The water sensitivity of the polyphosphazene can be varied from water-stable to water-sensitive by the choice of the substituent. In general, most R groups that are bound to the phosphazene backbone via a P—N bond are water sensitive and those that are bound via a P—O bond are water stable. Exceptions to the latter general rule are phosphazenes with glucosyl and glycolic and lactic acid esters substituents that are water-sensitive, even though these substituents are bound via a P—O bond. When [PR₂N]ₙ polymers react with water, NH₃, H₃PO₄ (or phosphates) and R—H are formed. Because NH₃ and H₃PO₄ and biologically compatible, the properties of RH determines whether water-erodible [PR₂N]ₙ polymers are biocompatible. Therefore, polyphosphazenes with glucosyl and glycolic and lactic acid esters substituents are biocompatible. Other biologically compatible substituents that give water-erodible phosphazenes include imidazolyl, glyceryl, and esters of amino acids, depsipeptides. With the various biocompatible R groups, hydrolysis of [PR₂N]ₙ takes days to several months. The water sensitivity can be tailored by synthesizing a polyphosphazene with two, or even three different substituents (of general form [PR₂N]ₓ[PRR′N]_{y}[PR′₂N]_{z}) and varying the relative amounts of the two substituents (x, y, and z). Polyphosphazenes have other potentially useful properties. They can be made into nanofibers and, depending on the R substituent, some have cell-adhesion properties.

In general, the compounds useful for antimicrobial applications, such as the treatment of bacterial and fungal infections, include silver(I) salts that are incorporated within the biodegradable nanomeric polymers including PLA, PGA, and PLGA are generally represented by formula 1 or by formula 2:

Ag^{⊕}X^{⊖} 1

Y^{⊖}Ag X^{⊖} 2

wherein X is represented by NO₃, OAc, SCN, BF₄, OTf, or SO₄ and wherein Y is represented by Li, Na, or K and X is represented by Cl, Br, or I.

The macrocyclic ligands that will be used to chelate to the silver salts represented by formula 1 are represented but not limited to formulas 3-6:

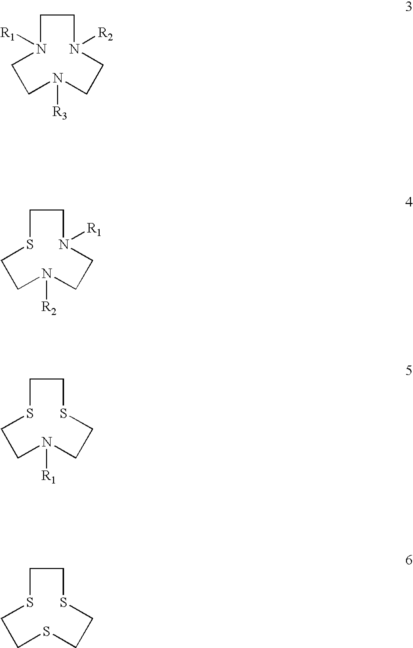

wherein each R can vary independently and can be a hydrogen atom, an alkyl such as but not limited to a methyl, an ether such as but not limited to methyl ethyl ether, an alcohol such as but not limited to ethanol, a carboxylic acid such as but not limited to acetic acid, an aryl such as but not limited to benzene, an amino acid such as but not limited to serine or threonine, or a peptide such as but not limited to luetinizing hormone. These R groups can be modified in order to increase the overall solubility of the complexes.

The N-heterocyclic carbenes that will be used to bind to Ag(I) are represented by but not limited to formulas 7-8:

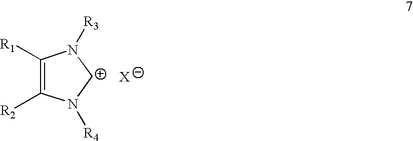

wherein R₁₋₂ can be independently or non-independently represented by a halide, a proton, an alkyl, an ether, an alcohol, a nitro, a cyano, or a carboxylic acid, wherein R₃₋₄ can be independently or non-independently represented by a hydrogen atom, an alkyl such as but not limited to a methyl, an ether such as but not limited to methyl ethyl ether, an alcohol such as but not limited to ethanol, a carboxylic acid such as but not limited to acetic acid, an aryl such as but not limited to benzene, an amino acid such as but not limited to serine or threonine, or a peptide such as but not limited to luetinizing hormone, and wherein X can be represented by NO₃, OAc, SCN, BF₄, OTf, SO₄, PF₆, BPh₄, Cl, Br, and I. These R groups can be modified for solubility purposes.

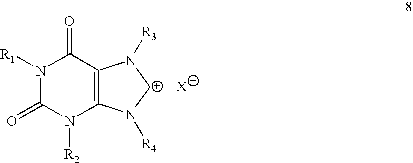

wherein R₁₋₄ can vary independently and can be a hydrogen atom, an alkyl such as but not limited to a methyl, an ether such as but not limited to methyl ethyl ether, an alcohol such as but not limited to ethanol, a carboxylic acid such as but not limited to acetic acid, an aryl such as but not limited to benzene, an amino acid such as but not limited to serine or threonine, or a peptide such as but not limited to luetinizing hormone, and wherein X can be represented by NO₃, OAc, SCN, BF₄, OTf, SO₄, PF₆, BPh₄, Cl, Br, and I. These R groups can be modified for solubility purposes.

In an example of preparing the nanoparticles, a relatively large amount of water is combined with a small amount of an organic solvent, and the silver(I) metal complexes incorporated within the nanoparticles will form in the organic portion of the mixture in the case of some nanoparticles and in the hydrophobic core of the nanoparticles in the case of other nanoparticles. Therefore, the selected silver(I) metal complexes may be hydrophobic. With this understanding, the silver(I) N-heterocyclic carbenes, as shown in formulas 9-13, have been prepared having hydrophobic substituent groups. The silver(I) N-heterocyclic carbenes, as shown in formulas 14 and 15, are further examples wherein R₁-R₃ represent the same or different hydrophobic substituent groups.

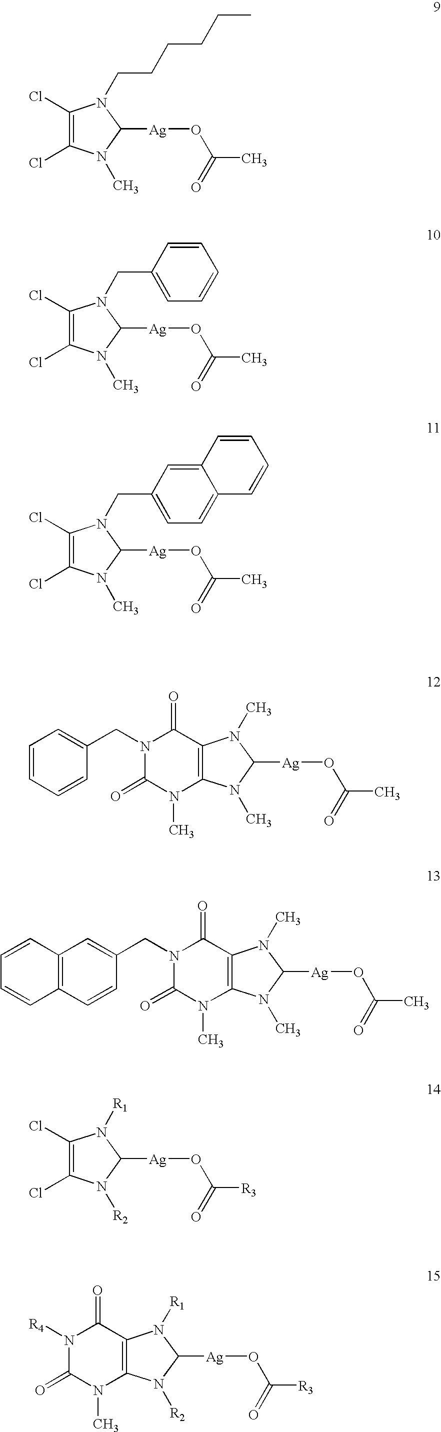

In one example, nanoparticles of compound 9 are incorporated within biodegradable polymers using both L-Tyrosine Polyphosphate (LTP) and 50:50 Poly[DL-lactide-co-glycolide], PLGA, LACTEL) polymers. The synthesis utilized a water-in-oil-in-water emulsion and a solvent evaporation technique. The formulations are given in Table 1. The initial emulsion was spun at 2000 rpm using a high speed mixer for 1 minute. After the addition of 10% PVP, the final emulsion was spun at 1600 rpm for 2 minutes. The organic solvent was allowed to evaporate under controlled conditions while constantly stirring for 5 hours.

TABLE 1
Formulations for nanoparticle preparation

| | Mass | PLGA | Mass | |
|---|---|---|---|---|
| LTP nanoparticles | (mg) | nanoparticles | (mg) | Solvent |
| L-Tyrosine | 264 | PLGA (50/50) | 270 | Chloroform |
| Polyphosphate | | | | |
| PEG-gChitosan | 3 | — | — | 1% acetic acid |
| Linear | 3 | — | — | Distilled Water |
| Polyethylenimine | | | | |
| Formula 9 | 30 | Formula 9 | 30 | Distilled Water |
| PVP (10%) | 10 | PVP (10%) | 10 | Distilled Water |

MIC testing of compounds 9 and 13 prior to loading in nanoparticles have been found to be more effective as an antimicrobial than other more water soluble silver metal complexes. Bacterial species tested include P. aeruginosa (PAO1, PAM57-15 and PAJG3), B. cepacia (PC783), B. multivorans (HI2229), B. cenocepacia (J2315), B. dolosa (ATTC BAA-246, AU3994, AU4459, AU4881, AU9248), M. tuberculosis and our control E. coli strains. The MIC90 for compound 9 against this panel of strains is 1 μg/mL (MIC range 1-2 μg/mL). Similarly, the MIC90 for compound 13 is 0.8 μg/mL (MIC range 0.8-2 μg/mL). The MIC of both compound 9 and compound 13 against the silver sensitive E. coli J53 is 1 μg/mL, while those against J53 containing the silver resistance plasmid pMG101 is >10 μg/mL. The MIC of both compound 9 and compound 13 against M. tuberculosis is 16 mg/mL

Another example of a silver(I) complex useful for the treatment of bacterial and fungal infections include silver(I) complex of formula 16 that is incorporated within the biodegradable nanomeric polymers.

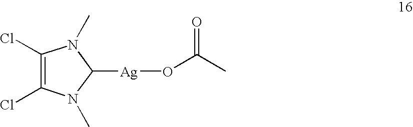

A further example of a silver(I) complex useful for the treatment of bacterial and fungal infections include silver(I) complex of formula 17 that is incorporated within the biodegradable nanomeric polymers. In order to maintain a concentration of the complex above the minimum inhibitory concentration (MIC) levels in the lungs, treatments should be administered throughout the day. To avoid this inconvenience, the use of inhaled nanospheres may be used to deliver the silver(I) complex of formula 17, which possesses potent antimicrobial properties, directly to the site of infection. This formulation may allow the slow release of the compound thereby creating a depot effect in chronically infected lungs. Sustained release of the compound also may have the advantage of decreased administration, which should lead to patient compliance.

In preparation of the silver(I) complex of formula 17, a 1-hexyl-3-methyl-4,5-dichloroimidazolium iodide salt precursor 1 was synthesized by the deprotonation of 4,5-dichloroimidazole with potassium hydroxide (KOH) followed by an alkylation with one equivalent of 1-bromohexane in acetonitrile. An excess amount of iodomethane was then added to the solution mixture, which was refluxed overnight. The crude oily product was stirred in diethyl ether resulting in a yellow powder in good yield (Scheme 1).

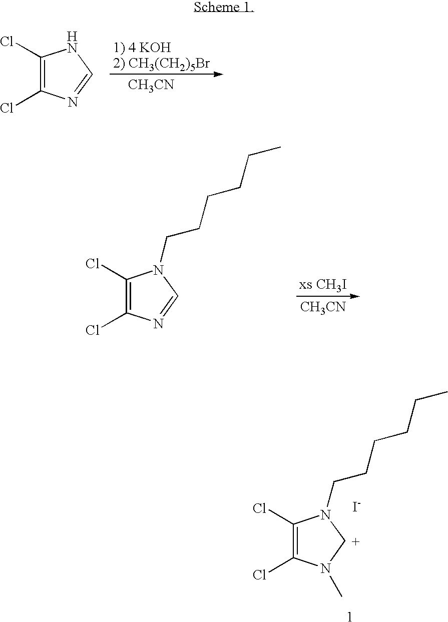

The ¹H and ¹³C NMR spectra of 1 are consistent with its molecular structure. In the ¹H NMR spectrum the imidazolium proton appears at 9.46 ppm. This shift is consistent with the general C2-H acidic proton shift of imidazolium salts (δ 8-10 ppm). The ¹³C NMR shift of the N—C—N sp² carbon, which later becomes the carbene center, appears at 136.3 ppm for 1. Individual crystals of 1, suitable for X-ray diffraction analysis, were obtained by evaporation from a concentrated solution of acetone. The molecular structure of the cationic portion of 1 is depicted in FIG. 1.

The in situ deprotonation of 1 with silver acetate in an approximately 1:2 molar ratio in dichloromethane, afforded the corresponding silver(I) complex of formula 17 in sufficient yield, as seen in reaction equation 1.

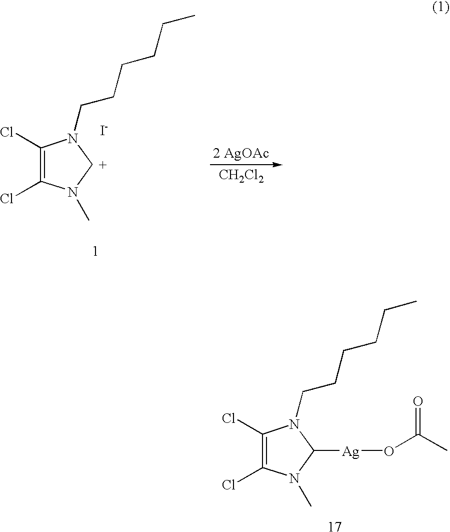

It appears that a significant feature of the ¹³C NMR spectrum of silver(I) complex of formula 17 is the signal at 179.6 ppm corresponding to the carbene carbon atom, which appears in the typical range of other NHC complexes of Ag(I), and the loss of the resonance at 136.3 ppm. The ¹H NMR spectrum shows a disappearance of the imidazolium proton signal at ca. 9 ppm, which further indicates the formation of the expected NHC silver acetate complex.

In one embodiment of the invention, silver(I) complex of formula 17 may be encapsulated into nanospheres formulated with L-tyrosine polyphosphate (LTP). LTP is synthesized from the amino acid L-tyrosine by linking the molecules with a peptide bond and coupling a phosphate onto the amino acid. The polymerization of repeating units results in L-tyrosine polyphosphates (LTP) with a molecular weight of 10 kDa and is classified as a ‘pseudo’ poly[amino acid]. LTP is soluble in most organic solvents, which allows for its easy formulation into nanospheres. Previous studies have shown films of LTP fully degrading in 7 days due to the presence of hydrolytically unstable phosphoester linkages in LTP's polymeric backbone. This degradation rate makes LTP an potential candidate for sustained drug delivery in biofilms without permanently accumulating into the lung tissue. Furthermore, the degradation products of LTP have been shown to result in an insignificant decrease in the local pH unlike other biomaterials such poly[lactic-co-glycolic acid] (PLGA). These probable degradation products of LTP include L-tyrosine based derivatives and phosphate ions that are nontoxic to the body along with alcohols.

The fabrication of LTP nanospheres encapsulating silver(I) complex of formula 17 may be accomplished using a water-in-oil-in-water emulsion. The external phase may consist of 10% polyvinyl pyrrolidone (PVP) and NaNO₃. LTP is utilized for about 88% of the nanosphere mass due to its ability to hydrolytically degrade within about 7 days, which gives the nanospheres a sustained delivery characteristic. Incorporating poly(ethylene glycol)-g-chitosan (PEG-g-CHN) and linear polyethyleneimine (LPEI) assists in stabilizing the emulsion during nanosphere formulation, thereby preventing aggregations. Due to the amphiphilic nature of the PEG-g-CHN and LPEI, these polymers enrich at the water-oil interface and prevent coalescence of the LTP droplets by steric repulsion. Using emulsion techniques, the encapsulation of low molecular weight drugs such as silver(I) complex of formula 17 may be difficult since the drugs can diffuse out of the oil phase and into the external water phase during the solvent evaporation. Previous attempts to encapsulate carbene complexes without the hydrophobic group resulted in undetectable amounts of our drug as detected by loading studies. Thus, the conjugation of the hexyl side chain and the ionic charge gradient created by the addition of the NaNO₃ in the external liquid phase are carefully designed to substantially confine silver(I) complex of formula 17 in the polymeric solution.

These encapsulation techniques have produced nanospheres with the loading of silver(I) complex of formula 17 at the therapeutic levels. In one embodiment of the invention, a target for silver(I) complex of formula 17 loading was about 2.5% of the total polymer weight to ensure optimal dosing while minimizing the delivery of polymer. The loading has been determined by quantifying the amount of silver extracted from LTP nanospheres via absorbance spectrophotography. The LTP nanospheres are formulated with about 10% (w/w) of silver(I) complex of formula 17; however, the actual loading is determined to be about 7.1%±1%. The encapsulation efficiency translates to about 71%±10%. This encapsulation of silver(I) complex of formula 17 may increase the drug's stability in water and biological systems, since silver carbene complexes are sensitive to moisture.

The method for encapsulating silver(I) complex of formula 17 yielded heterogeneous distribution of nanospheres with a mean diameter of approximately 1000 nanometers. Encapsulating low molecular weight drugs into nanospheres alleviates their quick clearance from tissues. In order to treat biofilms infection, nanospheres must be small enough and aerodynamic to navigate through 10 μm respiratory tracts, while large enough to deposit into the matrix of the biofilms. The shape, size, and morphology of our nanospheres have been determined using Scanning Electron Microscopy (SEM). The images obtained by SEM have shown a smooth surface morphology for both blank and silver(I) complex of formula 17-loaded LTP nanospheres. The diameter range of the nanospheres is between about 500 to about 5000 nm and the shape of all nanosphere formulations is substantially spherical as seen in FIGS. 2 and 3. The size of the LTP nanospheres was further quantified using a dynamic laser light scattering system and the results of the blank and silver(I) complex of formula 17-loaded LTP nanosphere diameter ranged between about 471 to about 2891 nm and between about 555 to about 1519 nm, respectively as seen in FIG. 4. Hence, these nanospheres are the proper size to be able to navigate through the respiratory tracts while still large enough to deposit into the matrix of the biofilms.

In order to provide a depot effect of sustained antimicrobial drug delivery, nanospheres may be configured to release the silver(I) complex of formula 17 over a period of one week. The LTP nanospheres have been shown to release all of the encapsulated silver(I) complex of formula 17 in 7 days under in vitro incubation in phosphate buffer saline (PBS), as shown in FIG. 5. The release profile of silver(I) complex of formula 17 from the LTP nanospheres has been determined by measuring the absorbance of precipitated silver obtained from the release supernatant of nanospheres suspended in buffer solution. The cumulative release after 7 days from 1 mg of LTP nanospheres is about 74 μg±10 μg, which is comparable to the loading data of about 71 μg±1 μg. Approximately 80% of the silver(I) complex of formula 17 is released within the first 2 days from the LTP nanospheres, which corresponds to about 60 μg and about 20 μg per 1 mg of nanosphere on days 1 and 2, respectively. This release rate from the nanospheres is a result of LTP's ability to hydrolytically degrade within 7 days.

LTP nanospheres have also been tested for the oral route of administration into to the lungs by nebulization. Approximately 5 mg of LTP nanospheres loaded with silver(I) complex of formula 17 were suspended in 1 ml of 10× phosphate buffer (without NaCl). Afterwards, the nanospheres were placed into the nebulization chamber, nebulized, and the vapor was collected directly into a 50 ml centrifuge tube. The collected condensation was observed by light microscopy and captured with a CCD camera. The nebulization of phosphate buffer alone was free of nanospheres, but LTP nanospheres readily passed through the nebulizer, as seen in FIG. 6. These results indicate that the oral route of administration for the LTP nanospheres is a viable method for localized delivery of silver(I) complex of formula 17 into the lungs.

Encapsulating silver(I) complex of formula 17 into degradable nanospheres may also provide a method for targeting microbes such as bacteria in biofilms. Targeting moieties such as peptides specific to the Pseudomonas aeruginosa bacteria can be easily incorporated into the nanosphere formulations. It has been discovered that toll-like receptors (TLR) that are specific to the lipopolysaccharide (LPS) on the pseudomonas aeruginosa bacterial biofilms could be attached to the surfaces of nanospheres.

In another embodiment of the invention, an initial evaluation of the in vivo antimicrobial effects of LTP nanospheres was conducted. In this evaluation, an amount of aerosolized silver(I) complex of formula 17-loaded and blank LTP nanospheres were delivered in a nose-only fashion to mice in a multi-dosing chamber. The mice received one dose of silver(I) complex of formula 17-loaded (about 36 mg) or a blank (about 34 mg) nanospheres one hour after inoculation with Pseudomonas aeruginosa followed by a second dose 24 hours after the first, as shown in FIG. 7A. It was noted that the animals exhibited substantially no significant abnormal behaviors from the high-concentration static exposure provided by the nebulizer, even after repeated dosing (about 2 mg silver(I) complex of formula 17/dose in the multi-dosing chamber, 15 minute exposure, daily) over a period of 2 days (data not shown). The mice were weighed daily and observed for a total of 72 hours after inoculation for survival. Treatment with silver(I) complex of formula 17-loaded LTP nanospheres resulted in an almost 20% survival advantage ( 12/16 silver(I) complex of formula 17-treated versus 9/16 blank, as seen in FIG. 7B) and the surviving animals demonstrated less weight loss compared with surviving animals treated with blank nanospheres, as seen in FIG. 7C.

In particular, the treatment protocol for the in vivo antimicrobial effects of LTP nanospheres includes the following procedures. Mice received treatment with nebulized blank or silver(I) complex of formula 17-loaded LTP nanospheres one hour after infection with P. aeruginosa PA M57-15, and again 24 h after the first treatment, until mice were evaluated on day 3 (72 h). B) Kaplan-Meier survival curves. The survival of mice treated with silver(I) complex of formula 17-loaded LTP nanospheres was greater than that of sham-treated animals (75% survival in 16 silver(I) complex of formula 17-treated mice versus 56% in 16 blank-treated mice). C) With regards to weight loss, the mice treated with silver(I) complex of formula 17-loaded LTP nanospheres lost less weight than animals treated with blank nanospheres. These results are displayed as mean % weight change from inoculation weight+/−SEM. There was substantially no statistical difference between groups at each time point.

To better quantify the antimicrobial effects of the nanospheres, the lungs and spleens of each animal were harvested for gross examination and quantitative bacteriology. The organs of animals that died were harvested within 1 hour after death. The lungs and spleens of survivors were harvested immediately after euthanasia at 72 hours after inoculation. The lungs from the majority of mice treated with silver(I) complex of formula 17-loaded LTP nanospheres appeared grossly normal (bright pink with a smooth surface). In contrast, the lungs from most of the mice that received blank LTP nanospheres appeared inflamed (red or dark pink with an enlarged volume). The spleens from both groups showed no significant morphological disparity. Treatment with silver(I) complex of formula 17-loaded LTP nanospheres resulted in a significant decrease in the bacterial burden in the lungs of surviving animals (3.6×10⁴+/−2.8×10⁴ silver(I) complex of formula 17 versus 5.3×10⁵+/−2.6×10⁵ blank, p<0.03, data from one animal is missing due to contamination of the plates, as seen in FIG. 8A). The bacterial counts in the lungs of animals that died were significantly higher than these in both groups and may represent post-mortem replication of bacteria (data not shown). The bacterial burden in the spleens of the surviving silver(I) complex of formula 17-treated animals was less than that of the sham-treated animals (1.3×10²+/−8.9×10¹ for SCC10 versus 5.7×10²+/−3.2×10² for blank, as seen in FIG. 8B), although the difference was not statistically significant (p=0.074). Survival is likely related to bacteremia as demonstrated by dissemination of bacteria to the spleen. Indeed, substantially all of the animals that died had bacteria recovered from their spleens. The animals treated with silver(I) complex of formula 17-loaded nanospheres had a significantly lower probability of dissemination of bacteria to the spleen (p<0.03, as seen in FIG. 8C), which may explain the survival advantage. These findings indicate that treatment with silver(I) complex of formula 17-loaded LTP nanospheres can effectively decrease the burden of bacteria in the lung, bacteremia, and likelihood of death in a Pseudomonas aeruginosa pneumonia model.

The term effective amount defines the dosage needed for proper treatment. The dosage will vary based on the silver(I) metal complex used and the physiological characteristics of the patient, and the nature and location of the cells, tissue, region of the body or other factors being treated. The type of drug administration will also vary depending on the nature and location of the bacterial and fungal infections being treated.

The method of treatment can be but is not limited to intravenous injection, intraperitoneal injection, inhalation, nebulization or oral ingestion. If the injection method is used, the drug can be dissolved in a suitable solvent. The choice solvent is typically a physiological saline solution. This solution can range from 0.5 to 1.0% sodium chloride in water because at this concentration the saline solution is of biological significance as it is isotonic with blood plasma. Another suitable solvent is dimethyl sulfoxide (DMSO). Other biologically acceptable solvents are also acceptable. The inhalation method will involve nebulization of the drug, as the drug will be inhaled as an aerosol. The oral ingestion method includes ingestion of the drug as a pill, capsule, caplet or tablet.

Formulation of the silver(I) metal complexes as a nanoparticle delivery system confers various clinical advantages. First, the formulation promotes slow leaching of the parent silver(I) metal complexes and active silver cation, thus providing a depot delivery of active drug if desired. This slow-release effect allows for increased dosing intervals and increased patient compliance. Furthermore, these particles can be taken up by alveolar macrophages and delivered to the systemic circulation. Previous studies have shown that aggregate particles in the size range of 1-5 μm can be phagocytized by macrophages, which subsequently migrate from the lung surface to the lymphatic system. Since the lymphatic system is intimately connected to the immune system as a whole, targeting of the silver(I) metal complexes drugs to the macrophages may offer benefits over traditional systemic delivery. If the immune system is targeted in this way, dose reduction is possible, yielding the same clinical outcomes as higher dosed oral or systemic type antimicrobials and eliminating potential dose-related side effects.

Based upon the foregoing disclosure, it should now be apparent that the use of metal compounds, including silver metal complexes, may be effective for antimicrobial applications, and in conjunction with biodegradable nanoparticles for the treatment of bacterial and fungal infections as described herein will carry out the objects set forth hereinabove. It is, therefore, to be understood that any variations evident fall within the scope of the claimed invention and thus, the selection of specific component elements can be determined without departing from the spirit of the invention herein disclosed and described.

1. A compound for antimicrobial applications, the compound comprising:
a metal complex incorporated into a polymeric nanoparticle.

2. The compound of claim 1, wherein the metal complex is a silver(I) metal complex.

3. (canceled)

4. The compound of claim 1, wherein the polymeric nanoparticle is biodegradable.

5. The compound of claim 1, wherein the metal complex is selected from the group consisting of a silver(I) salt, a silver(I) macrocyclic metal complex, a silver(I) N-heterocyclic carbene and mixtures thereof.

6. The compound of claim 5, wherein the silver(I) salt is selected from the group consisting of AgNO₃, AgOAc, AgSCN, AgBF₄, AgOTf and Ag₂SO₄.

7. The compound of claim 5, wherein the metal complex is silver(I) salt having the formula:
Y⁺AgX₂⁻, wherein Y is selected from the group consisting of Li⁺, Na⁺ and K⁺ and X is selected from the group consisting of Cl⁻, Br⁻ and I⁻.

8. The compound of claim 5, wherein the silver(I) macrocyclic metal complex comprises: 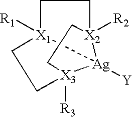
wherein each R is independently selected from the group consisting of a proton, an alkyl, an ether, an alcohol, a carboxylic acid, an aryl, an amino acid, a peptide, or null, wherein X₁, X₂ and X₃ are independently either sulfur or nitrogen, and when X₁, X₂ or X₃ is sulfur then R is null, wherein the macrocyclic ligand comprised of carbon, R₁₋₃, and X₁₋₃, represents L, wherein Y is selected from the group consisting of NO₃, OAc, SCN, BF₄, OTf, SO₄, Cl, Br, and I, or may represent L, and wherein Y represents L, then the counter anion is selected from the group consisting of NO₃⁻, OAc⁻, SCN⁻, BF₄⁻, OTf⁻, SO₄⁻, Cl⁻, Br⁻, and I⁻.

9. The compound of claim 5, wherein the silver(I) N-heterocyclic carbene comprises: 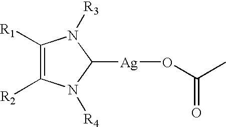
wherein R₁ and R₂ are selected from the group consisting of a halide, a proton, an alkyl, an ether, an alcohol, a nitro, a cyano, and a carboxylic acid, and wherein R₃ and R₄ are selected from the group consisting of a proton, an alkyl, an ether, an alcohol, a carboxylic acid, an aryl, an amino acid, and a peptide.

10. The compound of claim 5, wherein the silver(I) N-heterocyclic carbene comprises: 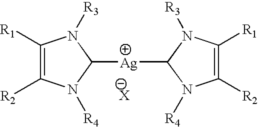
wherein R₁ and R₂ are selected from the group consisting of a halide, a proton, an alkyl, an ether, an alcohol, a nitro, a cyano, and a carboxylic acid, wherein R₃ and R₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

11. The compound of claim 5, wherein the silver(I) N-heterocyclic carbene comprises: 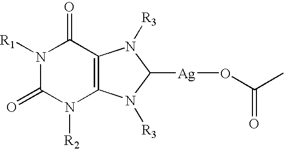
wherein R₁₋₄ are selected from the group consisting of a proton, an alkyl, an ether, an alcohol, a carboxylic acid, an aryl, an amino acid, and a peptide.

12. The compound of claim 5, wherein the silver(I) N-heterocyclic carbene comprises: 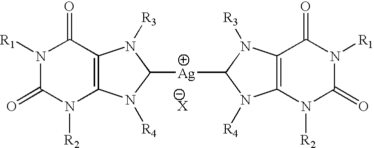
wherein R₁₋₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

13. A method of treating bacterial and fungal infections in a mammal, the method comprising the steps of:
administering an effective amount of a silver(I) metal salt incorporated into a biodegradable polymeric nanoparticle.

14. The method of claim 13, wherein the silver(I) salt is selected from the group consisting of AgNO₃, AgOAc, AgSCN, AgBF₄, AgOTf and Ag₂SO₄.

15. (canceled)

16. A method of treating bacterial and fungal infections in a mammal, the method comprising the steps of:
administering an effective amount of a macrocyclic silver(I) complex, the macrocyclic complex comprising: 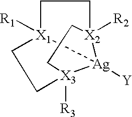
wherein each R is independently selected from the group consisting of a proton, an alkyl, an ether, an alcohol, a carboxylic acid, an aryl, an amino acid, a peptide, or null, wherein X₁, X₂ and X₃ are independently either sulfur or nitrogen, and when X₁, X₂ or X₃ is sulfur then R is null, wherein the macrocyclic ligand comprised of carbon, R₁₋₃, and X₁₋₃, represents L, wherein Y is selected from the group consisting of NO₃, OAc, SCN, BF₄, OTf, SO₄, Cl, Br, and I, or may represent L, and wherein Y represents L, then the counter anion is selected from the group consisting of NO₃⁻, OAc⁻, SCN⁻, BF₄⁻, OTf⁻, SO₄⁻, Cl⁻, Br⁻, and I⁻.

17. The method of claim 16, wherein the macrocyclic complex is incorporated into a biodegradable polymeric nanoparticle.

18. (canceled)

19. A method of treating bacterial and fungal infections in a mammal, the method comprising the steps of:
administering an effective amount of a N-heterocyclic silver(I) complex, the N-heterocyclic complex comprising: 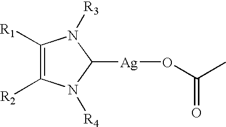
wherein R₁ and R₂ are selected from the group consisting of a halide, a proton, an alkyl, an ether, an alcohol, a nitro, a cyano, and a carboxylic acid, and wherein R₃ and R₄ are selected from the group consisting of a proton, an alkyl, an ether, an alcohol, a carboxylic acid, an aryl, an amino acid, and a peptide.

20. The method of claim 19, wherein the macrocyclic complex is incorporated into a biodegradable polymeric nanoparticle.

21. (canceled)

22. A method of treating bacterial and fungal infections in a mammal, the method comprising the steps of:
administering an effective amount of a N-heterocyclic silver(I) complex, the N-heterocyclic complex comprising: 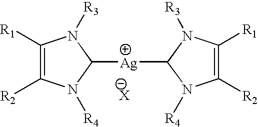
wherein R₁ and R₂ are selected from the group consisting of a halide, a proton, an alkyl, an ether, an alcohol, a nitro, a cyano, and a carboxylic acid, wherein R₃ and R₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

23. (canceled)

24. (canceled)

25. A method of treating bacterial and fungal infections in a mammal, the method comprising the steps of:
administering an effective amount of a N-heterocyclic silver(I) complex, the N-heterocyclic complex comprising: 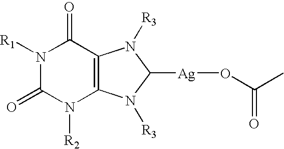
wherein R₁₋₄ are selected from the group consisting of a proton, an alkyl, an ether, an alcohol, a carboxylic acid, an aryl, an amino acid, and a peptide.

26. (canceled)

27. (canceled)

28. A method of treating bacterial and fungal infections in a mammal, the method comprising the steps of:
administering an effective amount of a N-heterocyclic silver(I) complex, the N-heterocyclic complex comprising: 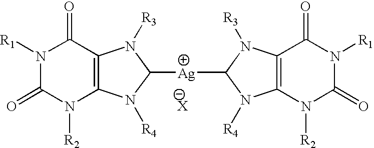
wherein R₁₋₄ are selected from the group consisting of a proton, an alkyl, an ether, an alcohol, a carboxylic acid, an aryl, an amino acid, and a peptide, and wherein X is selected from the group consisting of NO₃, OAc, SCN, BF₄, OTf, SO₄, Cl, Br, and I.

29. (canceled)

30. (canceled)
